(19) United States
(12) Patent Application Publication
(10) Pub. No.: US 2017/0273608 A1
(43) Pub. Date: Sep. 28, 2017

(54) IONIC BARRIER FOR FLOATING GATE IN VIVO BIOSENSORS

(71) Applicant: Ohio State Innovation Foundation, Columbus, OH (US)

(72) Inventors: Paul R. Berger, Columbus, OH (US); Anisha Ramesh, Hillsboro, OH (US)

(21) Appl. No.: 15/613,914

(22) Filed: Jun. 5, 2017

Related U.S. Application Data
(63) Continuation of application No. 13/624,197, filed on Sep. 21, 2012.
(60) Provisional application No. 61/537,723, filed on Sep. 22, 2011.

(51) Int. Cl. A61B 5/145; A61B 5/1473

(52) U.S. Cl. CPC A61B 5/14546; A61B 5/14735; G01N 27/4143

(57) ABSTRACT

An ion-sensitive sensor includes a dielectric layer comprising Al₂O₃ having a functionalized surface configured to bond with an analyte. The ion-sensitive sensor is immersed in an electrolytic solution containing a concentration of alkali ions. An electrode is arranged to apply an electric potential to the functionalized surface of the ion-sensitive sensor. In some embodiments the ion-sensitive sensor is an ion-sensitive silicon FET. In some embodiments the ion-sensitive sensor is an ion-sensitive polymer FET. In some embodiments, the electrode comprises a perforated gate metal layer disposed on the gate dielectric layer of an ion-sensitive FET, and the functionalized surface is disposed in openings of the perforated gate metal layer. In some embodiments the dielectric layer comprises a multi-layer dielectric stack including at least one Al₂O₃ layer. In some embodiments the dielectric layer is deposited by atomic layer deposition (ALD).

This application claims the benefit of U.S. Provisional Application No. 61/537,723 filed Sep. 22, 2011 entitled “IONIC BARRIER FOR FLOATING GATE IN VIVO BIOSENSORS”. U.S. Provisional Application No. 61/537,723 filed Sep. 22, 2011 entitled “IONIC BARRIER FOR FLOATING GATE IN VIVO BIOSENSORS” is incorporated by reference herein in its entirety.

### BACKGROUND

The following relates to the in vivo, ex vivo, and in vitro biological sensor (i.e. “biosensor”) arts, chemical sensor arts, and related arts.

Ion-selective field effect transistors (FETs) are known. See, e.g. Schöning et al., Analyst, 127, 1137 (2002). In such devices, the conventional gate electrode is replaced by an ion-sensitive layer in contact with an electrolytic solution. A reference electrode is immersed in or contacts the electrolyte to provide a reference potential, and this reference electrode defines the potential of the electrolyte. The gate voltage is the reference electrode potential modified by any charge accumulation or depletion at the ion-sensitive layer. Any such charge accumulation or depletion can induce charge in the FET channel, modifying the drain current and hence the operating characteristics of the ion-selective FET device. Some background on such devices is set forth in, e.g.: Schöning et al., Analyst, 127, 1137 (2002); Grieshaber et al., Sensors, 8, 1400 (2008). Such biosensors have been applied to different target applications, including glucose, pH, protein, and DNA detection and measurement. See, e.g. Piechotta et al., Biosensors and Bioelectronics, 21, 802 (2005); Chen et al., Appl. Phys. Lett., 89, 22351 (2006); Elibol et al., Appl. Phys. Lett., 92, 193904 (2008); Ouyang et al., Anal. Chem., 79, 1502 (2007); Star et al., Nano Letters, 3, 459 (2003); Gabl et al., Biosensors and Bioelectronics, 19, 615 (2004); Nicholson et al., Proceedings of the Institution of Mechanical Engineers, Part N: Journal of Nanoengineering and Nanosystems, 223, 149 (2010); Kim et al., Biosensors and Bioelectronics, 20, 69 (2004); Calleja et al., Ultramicroscopy, 105, 215 (2005); Li et al., Nano Letters, 4, 245 (2004).

One type of biosensor is a pH sensor. See, e.g. Schöning et at., Analyst, 127, 1137 (2002). In a pH sensor the ion-sensitive layer serving as the “gate” of the ion-selective FET is typical a SiO₂ layer or a double layer insulator of SiO₂—Si₃N₄, SiO₂—Al₂O₃ or SiO₂—Ta₂O₅, where the upper layer for the double insulator structures, i.e. Si₃N₄, Al₂O₃ and Ta₂O₅, typically serves as the sensitive material for pH-sensitive ion-sensitive FET devices. Id. In another pH sensor design (Reddy et al., Biomedical Microdevices, 13, 335 (2011)), the ion-sensitive layer is a single Al₂O₃ layer, which was found to provide improved pH sensitivity versus a SiO₂ layer, along with better long-term stability (as indicated by very small threshold voltage drift for 8 hours in a Robinson buffer at a near neutral pH=7.5). The improved pH sensitivity and robustness of the single Al₂O₃ layer as compared with SiO₂ was attributed to the higher dielectric constant (i.e., high-k) of Al₂O₃ and consequently thicker physical layer providing reduced gate leakage. Id.

An example of a biosensor is a protein biosensor, which is of importance in modern medicine for use in the early detection and diagnosis of disease, for instance cancer, See, e.g. Wee et al., Biosensors and Bioelectronics, 20, 1932 (2005); Arntz et al., Nanotechnology, 14, 86 (2003); Martin et al., Proteomics, 3, 11244 (2003); Abbott et al., Current Biology, 14, 2217 (2004). Different approaches for protein biosensors based on different semiconductor materials have been explored, such as AlGaN/GaN and carbon nanotubes. See, e.g., Gupta et al., Biosensors and Bioelectronics, 24, 505 (2008); Kang et al., Appl. Phys. Lett., 87, 023508 (2005); Kang et al., J. of Appl. Phys., 104, 031101 (2008); Gooding et al., J. Am. Chem., 125, 9006 (2003); Besteman et al., Nano Letters, 3, 727 (2003); Wang, Electroanalysis, 17, 7 (2005). Silicon (Si)-based protein biosensors have also been explored. See, e.g. Ouyang et al., Anal. Chem., 79, 1502 (2007); Veiseh et al., Biomedical Microdevices, 3, 45 (2001); Wang et al., Biosensors and Bioelectronics, 24, 162 (2008). Compared to the alternative material platforms, Si-based protein biosensors are low-cost and envisioned to be easily integrated onto a small chip atop a diagnostic needle complete with readout circuitry.

### BRIEF DESCRIPTION

In some illustrative embodiments disclosed as illustrative examples herein, a system comprises: an ion-sensitive sensor that includes a dielectric layer including Al₂O₃; an electrolytic solution in which the ion-sensitive sensor is immersed, the electrolytic solution containing a concentration of alkali ions, a surface of the dielectric layer of the ion-sensitive sensor being in contact with the electrolytic solution; and an electrode arranged to apply an electric potential to the surface of the the dielectric layer in contact with the electrolytic solution. In some embodiments the ion-sensitive sensor is an ion-sensitive silicon field effect transistor (FET). In some embodiments the ion-sensitive sensor is an ion-sensitive polymer FET. In some embodiments, the ion-sensitive sensor is a FET, the dielectric layer is the gate dielectric layer of the FET, and the electrode comprises a perforated gate metal layer disposed on the gate dielectric layer of the ion-sensitive FET, a functionalized surface being disposed in openings of the perforated gate metal layer. In some embodiments the dielectric layer comprises a multi-layer dielectric stack including at least one Al₂O₃ layer.

In some illustrative embodiments disclosed as illustrative examples herein, a method comprises: depositing a gate dielectric layer comprising Al₂O₃ on a substrate by atomic layer deposition (ALD) to form an ion-sensitive field effect transistor (FET); and modifying an exposed surface of the deposited gate dielectric layer to generate a functionalized gate dielectric surface configured to bond with an analyte. In some embodiments the method further comprises immersing the ion-sensitive FET with the functionalized gate dielectric surface in an electrolytic solution containing a concentration of alkali ions, and operating the ion-sensitive FET to measure concentration of the analyte in the electrolytic solution, the operating including biasing an electrode arranged to apply an electric potential to the functionalized gate dielectric surface of the ion-sensitive FET.

In some illustrative embodiments disclosed as illustrative examples herein, a sensor comprises: an ion-sensitive field effect transistor (FET) or capacitor that includes a dielectric layer comprising Al₂O₃, and a perforated metal layer disposed on the dielectric layer of the ion-sensitive FET or capacitor. The dielectric layer includes a functionalized surface configured to bond with an analyte, the functionalized surface being disposed in openings of the perforated metal layer. In some embodiments the functionalized surface is a functionalized Al₂O₃ surface. In some embodiments the ion-sensitive FET or capacitor is an ion sensitive FET, the dielectric layer comprising Al₂O₃ is the gate dielectric layer of the ion-sensitive FET, and the metal layer is a gate metal layer dispose on the gate dielectric layer of the ion-sensitive FET. In some embodiments the ion-sensitive FET is an ion-sensitive silicon FET. In some embodiments the ion-sensitive FET is an ion-sensitive polymer FET.

### BRIEF DESCRIPTION OF THE DRAWINGS

Unless otherwise noted, the drawings are not to scale or proportion. The drawings are provided only for purposes of illustrating preferred embodiments and are not to be construed as limiting.

FIG. 1 diagrammatically shows a protein biosensor employing an ion-sensitive field effect transistor (FET) as disclosed herein.

FIG. 2 diagrammatically shows a MOS capacitor used for testing oxide permeability by alkali ions as disclosed herein.

FIGS. 3 and 4 show Al₂O₃ gates with perforated gate metal where the perforations are holes (FIG. 3) or slots (FIG. 4).

FIGS. 5-14 plot results of tests described herein that were performed on MOS capacitors having the configuration shown in FIG. 2.

### DETAILED DESCRIPTION OF PREFERRED EMBODIMENTS

Although ion-sensitive FET devices can in principle serve as effective biosensors, their application in practice is more complex. The typical in vivo physiological environment contains Na⁺ and K⁺ ions that can be incorporated into the dielectric oxide of the ion-sensitive FET and contribute to mobile charge. See, e.g. Derbenwick, J. of Appl. Phys., 48, 1127 (1977); Kuhn et al., J. of Electrochem. Soc., 118, 966 (1971); Snow et al., J. of Appl. Phys., 36, 1664 (1965); Raider et al., J. of the Electrochem. Soc., 120, 425 (1973). These mobile ions are more deleterious than fixed charges due to gate oxide defects or interface charges, since the mobile ions shift within the active device depending upon voltage, causing a variable drift in the transistor threshold voltage, resulting in inaccurate in vivo operation for any electronics directly exposed to tissue and/or bodily fluids. Hence, it is recognized herein that a key feature needed for in vivo biosensors that are directly exposed to tissue or bodily fluids is impermeability to mobile alkali ions with stable transistor operation. As already noted, Si-based protein biosensors are low-cost and envisioned to be easily integrated onto a small chip atop a diagnostic needle complete with readout circuitry. However, Si-based protein biosensors suffer from long-term electrical drift and instability due to the diffusion of ions from high osmolarity biological buffers into the gate oxides

As disclosed herein, alkali ion penetration is a critical factor for threshold voltage instability in ion-sensitive FET biosensors using SiO₂ as the gate dielectric. As further disclosed herein, use of an Al₂O₃ gate dielectric us useful in a high ion concentration (0.15M) physiological buffer solution, because as shown herein the Al₂O₃ gate dielectric is impermeable to alkali ion penetration. This allows the future realization of low-cost Si-based in vivo biosensors or other Si-based biosensor for sensing analyte concentration in electrolytic solutions with high ion concentration (e.g., the illustrative 0.15M physiological butler solution).

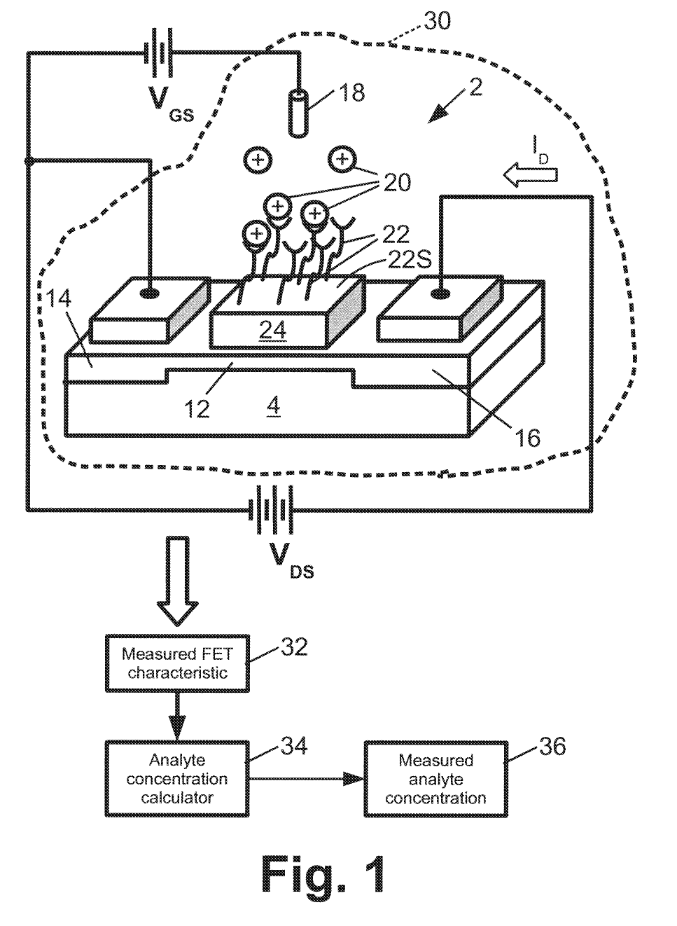

With reference to FIG. 1, a protein sensor includes an ion-sensitive field effect transistor (FET) 2 fabricated on a substrate 4 which may be a silicon substrate, a silicon-on-insulator (SOI) substrate (considered a silicon substrate herein), or other silicon-based substrate (e.g., alloyed with germanium). A sensing channel 12 connects a highly n-type doped (i.e. n⁺) source 14 and n⁺ drain 16 with a reference electrode 18. When a target protein 20 binds to a receptor 22 disposed on the gate dielectic layer 24 which in turn is disposed over (at least a portion of) the channel 12, it induces charges in the channel 12, causing a change in the current flow between the source 14 and drain 16. (It should be noted that in some embodiments the channel 12 is a topmost portion of the substrate 4 in which this charge is induced so as to form the channel 12 in an electrical sense; while in other embodiments the channel 12 may have some doping alloyed component, or other chemical anchor structural differentiation from the bulk substrate 4.) In conventional FET operation, a bias is applied to the gate electrode resulting in a charge of opposite polarity induced in the semiconductor channel due to the capacitive action of the gate-oxide-semiconductor structure. The accumulation of charge in the channel significantly raises its conductivity. The application of an additional voltage between the drain and source electrodes thus results in a current flow through the modified channel now with its voltage induced conductivity, thereby exhibiting gain in the drain current from the small gate voltage applied. In the ion-sensitive FET 2 of FIG. 1, the gate metal is replaced by a functionalized surface 22S of the gate dielectric layer 24 with analyte-specific affinity reagents (receptors 22), leaving the gate effectively “floating” in direct contact with an ionic solution 30 (diagrammatically indicated in FIG. 1) being tested. Binding of charged analytes 20 (protein to be detected, in the case of a protein sensor) to these surface receptors 22 results in a change in the charge induced in the channel 12, which manifests as a change in the drain current I_{D}. Proper tailoring of these receptors 22 restricts attachment of the analytes 20 only with the same conformation, so that the charged region of the analyte is in close proximity to the sensor (on the bottom) and all the attached analytes 20 induce an aggregate and additive gate voltage. Since a gate metal is absent in the ion-sensitive FET 2, a voltage is applied to the electrolyte 30 through the reference electrode 18 to shift the baseline transistor bias condition and maximize transistor gain.

Receptors 22 for measuring the protein streptavidin are described here as an illustrative example. Streptavidin is a tetrameric protein expressed more fully as Streptomyces avidinii. It is comprised of four identical subunits, each of which bind onto a complementary biotin molecule. It has an extraordinarily high affinity for biotin (also known as vitamin B7). The dissociation constant (K_{d}) of the biotin-streptavidin complex is on the order of about 10⁻¹⁴ mol/L. The high affinity of the noncovalent interaction between biotin and streptavidin forms the basis for many diagnostic assays that require the formation of an irreversible and specific linkage between biological macromolecules. Among the most common uses of streptavidin-biotin are the purification, or detection, of various proteins. The strong streptavidin-biotin bond can be used to attach various biomolecules to one another, or onto a solid support. Harsh conditions are needed to break the streptavidin-biotin interaction, which often denatures the protein of interest being purified. However, it has been shown that a short incubation in water above 70° C. will reversibly break the interaction without denaturing streptavidin, allowing re-use of the streptavidin solid support. The strong affinity between these two molecules, and its high degree of characterization, make it an ideal test bed for bioFET platforms. The affinity of streptavidin to the Al-bond on the surface Al₂O₃ gate dielectric provides an anchor point for the bioreceptor molecule. This can be applied by dip-coating, although orientation will be random and all areas may be coated, without significant selectivity. Alternatively, a nanometer-scale patterning method may be used to print Streptavidin on the surface of the bioFET channel. Streptavidin printing may enhance the functionality of the bioFET by tailoring the bioreceptor attachments. Nanopatterning places a single protein in a specific location by creating patterns on the order of nanometers, the same size as a protein, and is used in cell adhesion and signal transduction because of their smaller size. Nanopatterned surfaces for cell attachment have been fabricated by colloidal lithography, polymer demixing, and copolymer formation. These methods provide nanometer-scale topography. Electron-beam lithography (EBL) and a dry etching process can be used to control the scale and the shape of the patterns precisely on the bioFET channel. Protein on the surface can be stimulated by the nanometer-scale topography and analytes can be aligned along line and space patterns. The foregoing is merely an example, and the receptors 22 may in general be any molecule or macromolecule that selectively binds to an analyte organic molecule, an analyte toxic chemical of interest, or other so forth.

When the gate-source voltage (V_{GS}) is greater than the drain-source voltage (V_{DS}) the transistor operates in the linear region and the drain current-voltage relationship is given by

$${I}_{D} = μ {C}_{ox} \frac{W}{L} ( {V}_{GS} - {V}_{T} - \frac{1}{2} {V}_{DS} ) {V}_{DS} .$$

As the drain-source voltage is increased and exceeds V_{GS}−V_{T}, the device enters saturation and the drain current-voltage relation is given by

$${I}_{D} = \frac{1}{2} μ {C}_{ox} \frac{W}{L} {( {V}_{GS} - {V}_{T} )}^{2} .$$

Here, μ is the electron/hole mobility, Cₒₓ is the oxide capacitance given by

$${C}_{ox} = \frac{ɛ A}{{t}_{ox}} ,$$

W and L are the width and length of the gate, ε is the oxide permittivity, A is gate area, tₒₓ is oxide thickness and V_{T} is the threshold voltage. The threshold voltage is the minimum gate voltage to turn on the transistor and is given by

$${V}_{T} = ( {φ}_{ms} - \frac{{Q}_{f} + {Q}_{m}}{{C}_{ox}} ) + 2 {ψ}_{B} + \frac{\sqrt{4 {ɛ}_{S} {qN}_{A} {ψ}_{B}}}{{C}_{ox}}$$

where Φₘₛ is the work function difference between the metal and semiconductor, ψ_{B} is a potential energy controlled by the doping density, εₛ is the silicon permittivity, and N_{A} is the substrate doping concentration. Q_{f} is the fixed oxide charge introduced in the oxide during growth and is constant for a device. Qₘ is the mobile ion charge.

This mobile charge Qₘ impacts operation of the ion-sensitive FET 2. It is clear from the foregoing that changes in Qₘ result in changes in device threshold voltage and hence output current of the device. This will conflict with changes due to adsorbed protein analyte 20 and result in erroneous operation. For biosensors or other ion-sensitive FET devices designed to measure an analyte (excluding pH), the mobile charge Qₘ due to alkali ions in the electrolytic solution is a potentially a source of substantial error. Most formulations of the analyte-sensitive surface 22S of the gate dielectric layer 24 are likely to bind or release hydrogren (and/or hydroxide) ions to some extent, and hence the device characteristics are sensitive to pH. Nonetheless, this pH-dependent surface charge can be remediated by suitable calibration, and such calibration is aided in the case of in vivo measurements by tissue pH being relatively close to neutral, e.g. around 6.0-7.5. However, the additional effect of mobile charge Qₘ in the form of alkali ions permeating into the insulator produces a voltage- and time-dependent effect that is more difficult to compensate. Unlike the case for a pH sensor, there is no expectation that the mobile charge Qₘ will be correlated with the analyte concentration in the electrolytic solution.

As disclosed here, the use of an Al₂O₃ layer as the gate dielectric layer 24 provides an effective ion barrier. By using an Al₂O₃ layer as the gate dielectric layer 24 in combination with a suitable analyte-sensitive surface 22S (which may include discrete analyte-specific receptors 22 as shown, or alternatively may not include discrete analyte-specific receptors but instead have a chemical composition that is adsorptive for the analyte 20), the measured FET electrical characteristic 32 provides a useful input that can be analyzed by an analyte concentration calculator 34 (e.g., suitably embodied by a computer, microprocessor, or other electronic data processing device) compute and output an analyte concentration measurement 36.

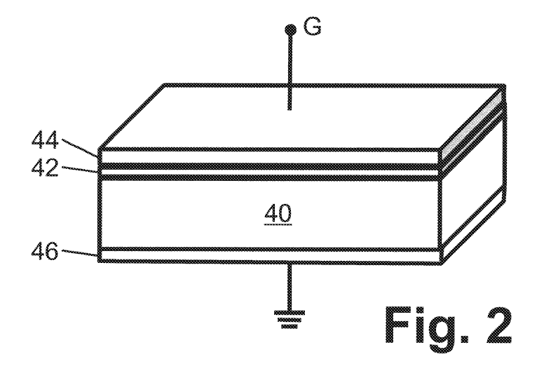

With reference to FIG. 2, a tractable model for a metal-oxide-semiconductor field effect transistor (MOSFET, where “oxide” here is not limited to SiO₂), is a simple MOS capacitor that can be effectively used to determine the presence of mobile ions, such as sodium (Na⁺) ions, in the oxide. The typical structure of a MOS capacitor is shown in FIG. 2, and includes a p-type silicon (p-Si) substrate 40, a dielectric oxide layer 42, a (front-side) metal contact layer 44 disposed over the oxide 42 and electrically connected with a gate (G), and a hack-side metal contact layer 46 disposed over the back-side of the substrate 40 and electrically connected to circuit ground. The dielectric oxide layer 42 is either aluminum oxide (Al₂O₃) deposited by atomic layer deposition (ALD), or thermally grown silicon oxide (SiO₂). In the Al₂O₃ samples, the atomic layer deposition (ALD) of aluminum oxide was carried out with trimethylaluminum (TMA) and water as precursors at 300° C. using a Picosun Sunale™ reactor. ALD is a layer-by-layer deposition method relying on self-limiting surface reactions to obtain atomic layer control of deposition. An advantage of ALD is precise thickness control at the Angstrom or monolayer level. The self-limiting aspect of ALD leads to excellent step coverage and conformal deposition on high aspect ratio structures. The silicon substrates 40 used were moderately doped (˜10¹⁶ cm⁻³) p-type silicon wafers. Prior to deposition, the silicon wafers were cleaned using a standard clean process consisting of RCA1 (1NH₄OH:1H₂O₂:5 de-ionized (DI) H₂O at 70° C. for 10 minutes) and RCA2 (1HCl:1H₂O₂:5 DI H₂O at 70° C. for 10 minutes). This was followed by a 1 minute dip in 1HF:10 DI and a 1 minute DI H₂O rinse. The ALD pulsing sequence for one cycle was 0.1 second per TMA pulse, 4 seconds per N₂ purge, 0.1 second per H₂O pulse, and 4 seconds per N₂ purge. Typical ALD deposition rates of 0.8 Å/cycle were obtained. The samples were then subjected to various anneals to determine the optimum anneal condition with minimal hysteresis and interface state density. The various anneal conditions used were 450° C. in forming gas (10% H₂, 90% N₂), 600° C. in oxygen ambient and 700, 800 and 900° C. in nitrogen ambient. Aluminum metal was deposited on the topside and patterned by photolithography and lift-off to obtain square electrodes with various areas of 275×275, 550×550, 1100×1100, 1650×1650 and 2200×2200 μm². The square electrodes were designed additionally with holes and slots to permit various levels of ion permeation and a control electrode was included with no holes. Finally aluminum metal was deposited on the backside of the wafer to complete the capacitor fabrication. This was followed by a post-metallization anneal at 450° C. for 10 min. in nitrogen ambient.

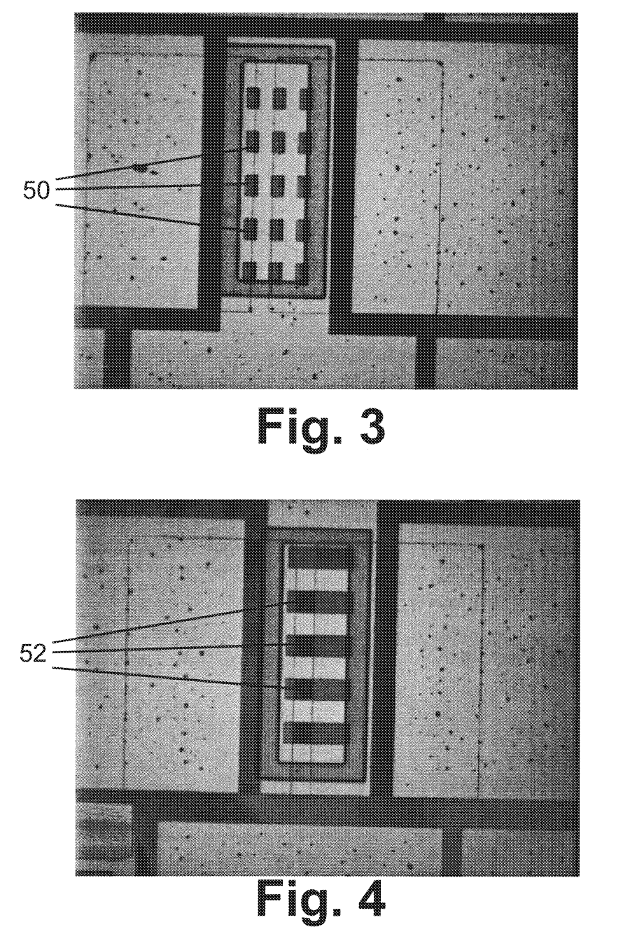

With reference to FIGS. 3 and 4, the hole and slot configurations for the square electrodes designed with holes or slots to permit various levels of ion permeation are shown. FIG. 3 shows an Al₂O₃ gate with perforated gate metal where the perforations are holes 50. FIG. 4 shows an Al₂O₃ gate with perforated gate metal where the perforations are slots 52. In both FIGS. 3 and 4, the gate test area has a width/length ratio of 10:1 with length 25 microns. An advantage of this approach is that a gate voltage can be applied directly to the gate (since there is a metal gate deposited on the Al₂O₃ (or SiO₂) insulator) but the gate is still sensitive to analyte ions (via the analyte-sensitive surface 22S of the gate dielectric layer 24 exposed by the holes 50 or slots 52). In embodiments employing a perforated gate metal layer disposed on the gate dielectric layer 24, the reference electrode 18 shown in FIG. 1 is optionally omitted.

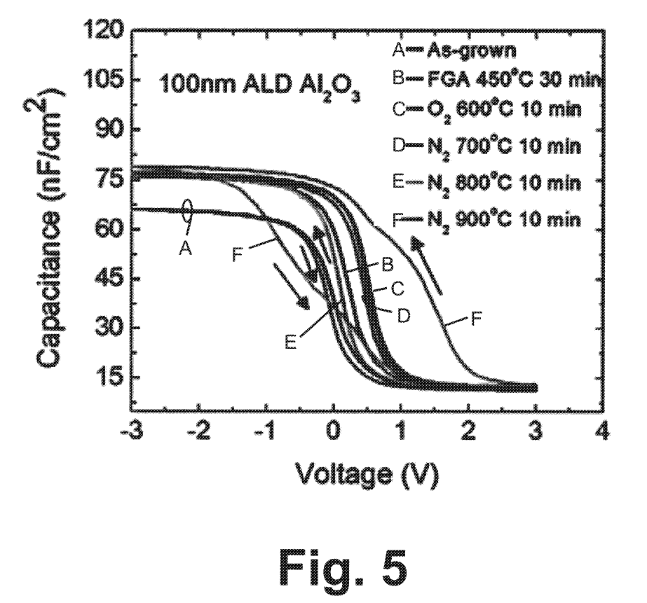

With reference to FIGS. 5-8, the quality of the oxide layer 42 of each test capacitor was characterized by hysteresis and multi-frequency capacitance-voltage measurements using an HP 4284 LCR meter, Hysteresis characteristics were obtained by sweeping the capacitor from depletion to accumulation and then reversing the sweep direction.

FIG. 5 shows the hysteresis characteristics obtained for samples with a 100 ALD-grown Al₂O₃ oxide layer subjected to various anneal conditions. All measurements were done at 100 kHz frequency. As-grown and low temperature forming gas annealed (FGA) samples show a hysteresis of 120 mV due to slow traps in the oxide. After annealing between 600 to 800° C., the oxide traps are reduced and no hysteresis is observed. Annealing at 900° C. results in a large hysteresis indicative of the formation of a large number of oxide traps as the oxide is annealed at temperatures above the crystallization temperature (850° C.). Ellipsometry was used to measure the oxide thickness. For the comparative study between ALD Al₂O₃ and thermal. SiO₂, a target thickness of 100 nm was chosen. As-grown Al₂O₃ was measured to be 103 nm. After annealing up to 800° C. the thickness reduced to 101 nm while annealing at 900° C. resulted in a larger thickness reduction down to 93 nm. The dielectric constant for the annealed samples is calculated to be 8.65 from C-V measurements.

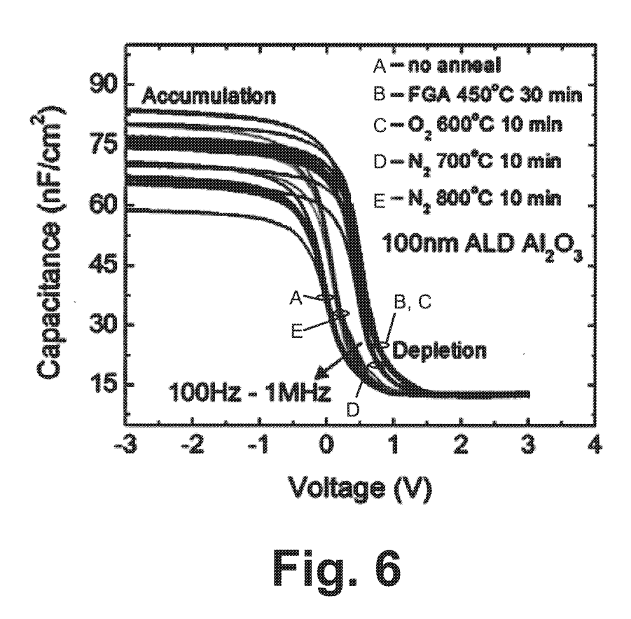

FIG. 6 shows multi-frequency capacitance-voltage (C-V) measurements for ALD Al₂O₃ under various anneal conditions. It should be noted that the drop in accumulation capacitance at a frequency of 1 MHz is due to the series resistance. Frequency dispersion in the depletion region is due to a frequency dispersive contribution to capacitance by interface traps which decrease with increasing frequency. Negligible dispersion is observed for all samples except for the 800° C. anneal sample. This correlates with an order of magnitude increase in interface density from ˜10¹⁰ cm⁻² eV⁻¹ for anneals at 700° C. to ˜10¹¹ cm⁻² eV⁻¹ range for anneals at 800° C. Thus, annealing at 700° C. in nitrogen ambient was found to be the optimal condition and was used for all the subsequent ALD Al₂O₃ samples used in this study.

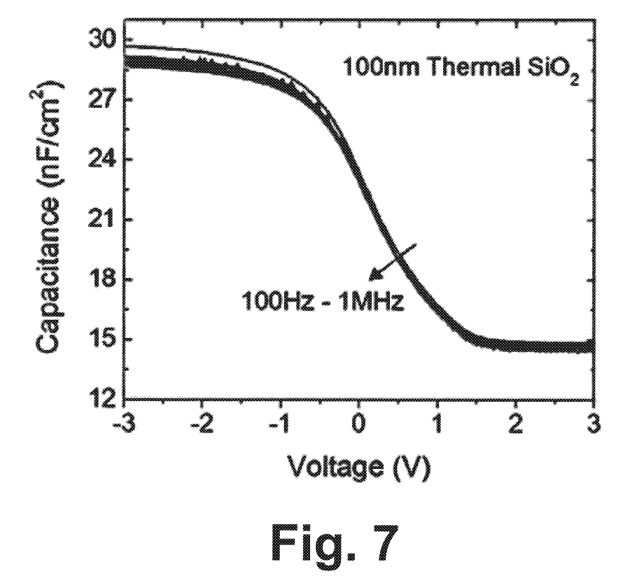

With reference to FIG. 7, thermally grown silicon oxide (SiO₂) was used as the control sample. The sample was prepared using the same p-doped substrate and wafer cleaning procedure as described above for ALD Al₂O₃. Dry silicon oxide was grown in an atmospheric tube furnace at 1050° C. with an oxygen ambient followed by a 20 minute nitrogen anneal at the same temperature. Multi-frequency C-V curves for SiO₂ indicate a good oxide quality with negligible frequency dispersion due to interface states, as evidenced by the results of FIG. 7. The oxide thickness was measured to be 116 nm with a calculated dielectric constant of 3.8.

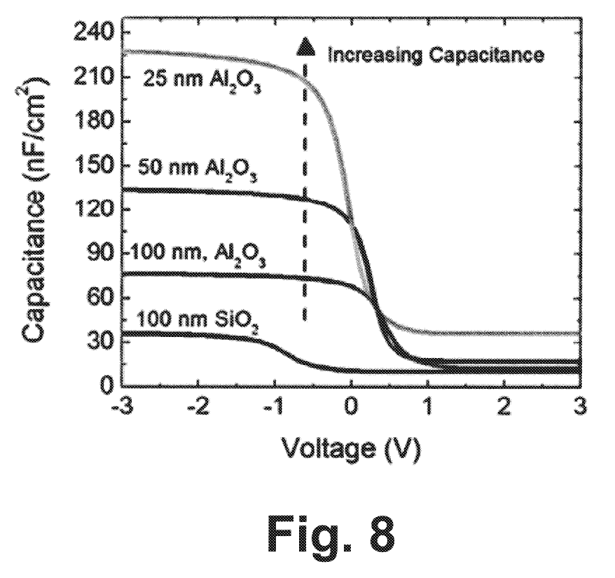

With reference to FIG. 8, reducing the oxide thickness further increases the capacitance and hence the sensitivity of a potential biosensor. The MOSFET channel current is directly proportional to the oxide capacitance,

$${C}_{ox} = \frac{ɛ A}{{t}_{ox}}$$

so that increasing the dielectric constant (ε) (using high-k dielectrics such as Al₂O₃) while concurrently reducing the oxide thickness (tₒₓ) provides a large sensitivity boost, which is advantageous for biosensing applications. MOS capacitors using Al₂O₃ as their dielectric and with reduced thicknesses were obtained by repeating the ALD process and reducing the number of cycles to obtain samples with target oxide thicknesses of 50, 25 and 10 nm, in addition to the 100 nm sample. The measured oxide thickness values using ellipsometry were 52, 30 and 12 nm, respectively. The effect of increased dielectric constant and reducing oxide thickness is illustrated in FIG. 8, where C-V plots (swept from depletion to accumulation and back) obtained from MOS capacitors formed with various Al₂O₃ oxide thicknesses and SiO₂ as the gate dielectric are juxtaposed. Excellent dielectric properties are observed for all ALD oxides with no observable hysteresis.

The in vivo physiological environment can be simulated by conducting experiments in physiological buffer solutions (pH 7.4, 0.15M Na⁺, K⁺). Natural in vivo protein environments contain comparable concentrations of alkali ions at a similar pH. Hence, impermeability of ions or immunity of transistor electrical response to these environments serves as a viable proof of applicability of Si-based FET sensors for in-vivo measurements or other (e.g., in vitro) measurements in which the ion-sensitive surface 22S is directly exposed to tissue and/or bodily fluids.

Permeation of mobile charges into the oxide can be quantified using the triangular voltage sweep (TVS) method. The TVS technique is based upon measuring the charge flow through the oxide at an elevated temperature in response to an applied time-varying voltage. See D. K. Schroder, Semiconductor Material and Device Characterization, (New York, Wiley, 2006), p. 340. in tests reported herein, the MOS sample was heated to a temperature (˜250° C.) where the mobile ions have sufficient thermal energy, and thus mobility, to respond to an applied bias. The MOS capacitor was stressed for 5 minutes at a voltage that generates about 1 MV/cm electric field across the oxide. This moves all the mobile ions to the capacitor plate charged with the opposite polarity. A triangular voltage ramp is subsequently applied to the gate of the capacitor. The ramp frequency should be slow enough so that the ions can drift through the oxide. Hence, a quasi-static capacitance-voltage C-V measurement is performed. This generates a displacement current in the capacitor. As the voltage crosses from positive to negative or negative to positive, a peak in the measured capacitance is observed. The capacitor is next stressed at an opposite polarity bias and a reverse voltage sweep is applied. The capacitance is obtained by measuring the charge flow (ΔQ) through the oxide when a time varying voltage is applied (ΔV) given by ΔQ/ΔV. The peaks in the two sweep directions may not be identical since the ions are at different interfaces (metal-oxide, oxide-semiconductor) after stressing at two different polarities. Next, a high frequency C-V measurement is performed, where the ions do not have sufficient time to respond, and no significant peak due to mobile ions is observed. Using this as the baseline, the area between these two curves (high frequency and low frequency) is determined by integration to obtain the mobile ion charge density within the oxide. Finally, MOS capacitors with ALD Al₂O₃ and thermal SiO₂ gate dielectrics were soaked in the physiological buffer solution for varying amounts of time and subsequently measured by the TVS technique.

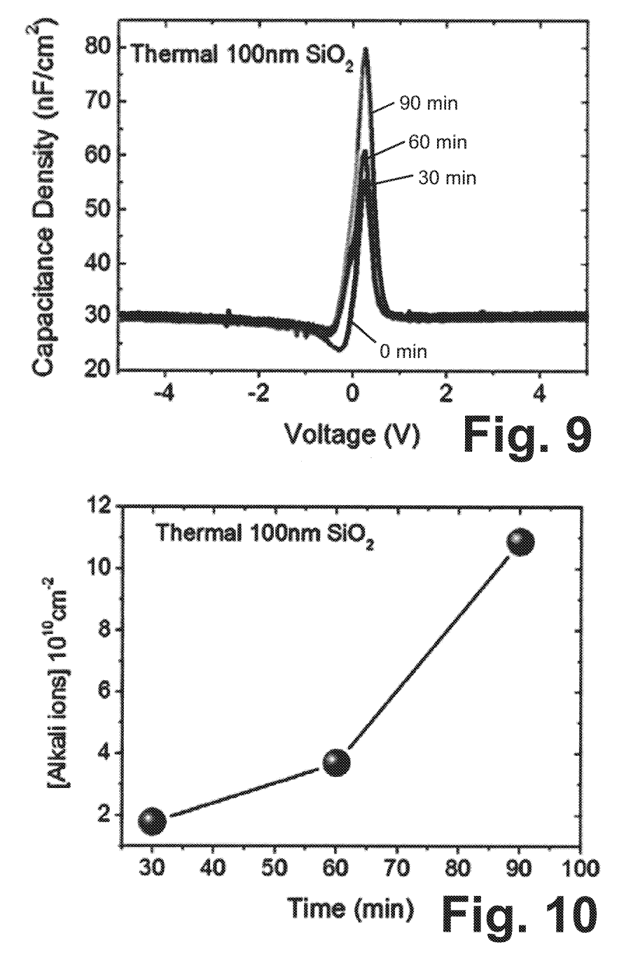

With reference to FIGS. 9 and 10, results of the alkali ion permeation into the oxide films of the test capacitors are shown. FIG. 9 shows the result of TVS measurements for a typical 100 nm SiO₂ MOS capacitor at 250° C. Ramp rates of 0.5 V/sec were used for all the measurements in this study. TVS measurements were conducted prior to dipping in the physiological buffer solution and after soaking in the physiological buffer solution for 30 min, 60 min, and 90 min. It should be noted that thermal SiO₂ shows a mobile ion peak prior to soaking in the physiological buffer solution. This is due to incorporation of some alkali ion contamination from the tube furnace during thermal oxidation. Additionally, as the soak time in the physiological buffer solution is increased, a clear linear increase in the mobile ion peak is observed. This indicates significant penetration of ions from the physiological buffer solution into the SiO₂ oxide. The area between consecutive curves quantifies the increased mobile charge (alkali ions) after each soak and is determined by numerical integration. TABLE 1 tabulates, and FIG. 10 plots, the increase in alkali ion penetration into SiO₂ MOS capacitors with increasing soak times in the physiological buffer solution.

TABLE 1
Relationship between increased alkali ion concentration
into thermal SiO₂ oxide (~100 nm) and PBS soak times

Time (min)

| | 0 | 30 | 60 | 90 |
|---|---|---|---|---|
| Δ[Alkali ions] | 0 | 1.77 | 3.69 | 10.87 |
| (×10¹⁰ cm⁻²) | | | | |

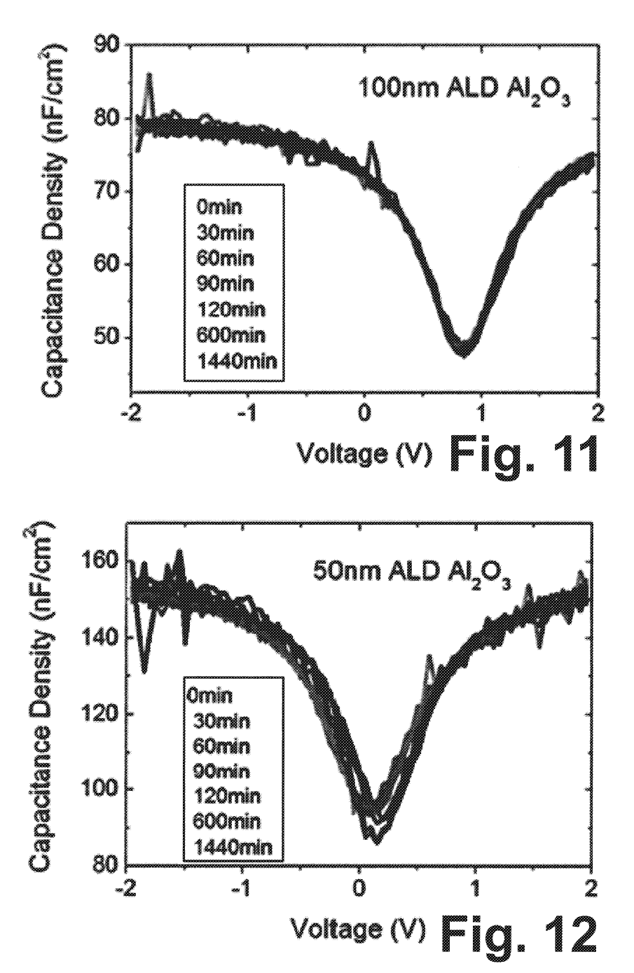

With reference to FIG. 11, the experiment was then repeated with a 100 nm thick ALD Al₂O₃ gate dielectric. The results are depicted in FIG. 11. No response due to alkali ion penetration is observed. The MOS device was next soaked for longer intervals of time up to 24 hours and the immunity to alkali ions penetration was confirmed for all time durations studied here. The three gate electrode topologies, holes (FIG. 3), slots (FIG. 4), and no holes (i.e., a continuous gate metallization completely covering the oxide layer 42—this serves as a reference since with full coverage no alkali ions should permeate into the oxide layer 42) also showed no measurable differences either (not shown here).

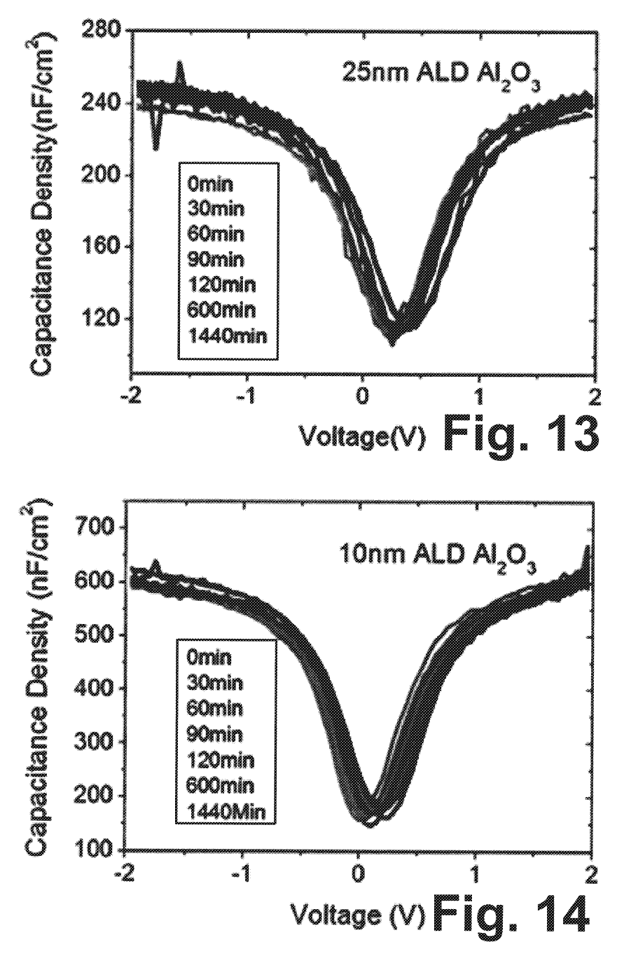

With reference to FIGS. 12-14, reduction in oxide thickness provides an additional benefit of increasing capacitance, hence increased sensitivity to analyte charge. This is particularly useful due to the low signal typically generated in such sensors and the exponentially increasing signal with decreasing thickness. Hence, MOS capacitors with reduced ALD Al₂O₃ oxide thicknesses (as compared with the nominal 100 nm Al₂O₃ samples shown in FIG. 11) were also fabricated and soaked in the physiological buffer solution as described above. TVS measurements were performed to test alkali ion penetration into these oxides. FIGS. 12, 13, and 14 depict the TVS measurement results for 50 nm, 25 nm, 10 nm Al₂O₃ thickness samples, respectively. No mobile ion response is observed for soak times in the physiological buffer solution of up to 24 hours for any of these thinner Al₂O₃ oxide thicknesses.

Silicon based protein biosensors directly exposed to tissue and/or bodily fluids suffer from long-term electrical drifting and instability due to the contamination of alkali ions from high osmolarity biological buffers. Their long-term stability and biocompatibility is of great concern which requires significant improvements for clinical use. As disclosed herein, a low-cost Si based MOS capacitor with a high-k Al₂O₃ dielectric deposited by ALD has been fabricated. The disclosed high-k dielectric layers not only prevent alkali ions diffusion from high osmolarity biological buffers into the gate oxides but also result in enhanced device sensitivity due to increased electrostatic coupling. Si-based ALD Al₂O₃ MOS capacitors show no measurable peak before and after soaking in the physiological buffer solution up to 24 hours, indicating no alkali ions penetration for various tested oxide thicknesses of 100 nm, 50 nm, 25 nm, 10 nm.

While ALD deposited Al₂O₃ has been shown by the foregoing experiments to provide alkali ion impermeability for the oxide of the ion-sensitive FET 2, other high-k oxides are expected to provide similar benefits, especially when deposited by ALD which produces films with low porosity. Various single layers, or multi-layer high-k dielectric stacks, are contemplated, such as combinations of Al₂O₃, hafnium silicate, zirconium silicate, hafnium dioxide (HfO₂), zirconium dioxide, tantalum oxide (e.g. Ta₂O₅), titanium dioxide (TiO₂), or combinations thereof, deposited by ALD creating ultrathin alternating layers, preferably toggling between materials to provide the maximum of chemical potential for trapping the unwanted ions and simultaneously providing high permittivities. The high-k material for use as the gate of the biosensor should satisfy requirements such as: good thermal stability in contact with Si so as to prevent the formation of a parasitic SiOₓ interfacial layer leading to lower “effective” permittivity or the formation of undesired silicide layers; low density of intrinsic defects at the Si/dielectric interface and in the bulk of the material so as to provide high mobility of charge carriers in the channel and sufficient gate dielectric lifetime; and sufficiently large energy band gap so as to provide high energy barriers at the Si/dielectric and metal gate/dielectric interfaces in order to reduce the leakage current flowing through the structure.

Moreover, while the disclosed alkali ion-impermeable oxide is disclosed in the context of an illustrative a Si-based ion-sensitive FET 2, it is contemplated to employ a bio-sustainable sensor including π-conjugated organic semiconductor active regions, such as a polymer field effect transistor (PFET), for example with standard regioregular poly (3-hexylthiophene) (RR-P3HT) channels. Conjugated semiconductor based electronics are 100% carbon based, in concert with the human body. So, the long-term rejection of man-made implants or biosensors is expected to be minimal. In order to improve the sensitivity and make biocompatibility biosensors, a variety of methods may be employed to boost the sensitivity of the polymer bioFET, including print ion-gel gate dielectrics for thin-film transistors on plastic and alternate conjugated polymers for high mobility channels, such as solution processable triisopropylsilyl pentacene (TIPSpentacene). Ion gel is a special class of solid polymer electrolytes which can serve as high-capacitance gate dielectrics. The faster polarization response is a manifestation of both the very large concentration and mobility of ionic species in the gels. An aerosol jet printing technique may be employed to print ion-gel on the channel of polymer bioFET to improve the sensitivity of polymer bioFET. Ion-gel dielectric is promising for flexible electronics applications by virtue of their large capacitance, printability and suitable frequency response. Combinations of ion-gel dielectrics with ion barrier Al₂O₃ are contemplated, and atomic layer deposition (ALD) is gentle enough (and is performed at sufficiently low temperature) to be combined with soft carbon based materials. Organic semiconductors, such as 6,13-bis(triisopropylsilylethynyl) (TIPS) pentacene, have been found to exhibit a very high charge carrier mobility (>1 cm² V⁻¹ S⁻¹) because the molecules arrange into a well-organized polycrystalline structure. Thus, a TIPS pentacene based polymer bioFET is contemplated, and other solution processable organic material is suitably applied to improve the mobility, consequently improving the sensitivity.

The preferred embodiments have been described. Obviously, modifications and alterations will occur to others upon reading and understanding the preceding detailed description. It is intended that the invention be construed as including all such modifications and alterations insofar as they come within the scope of the appended claims or the equivalents thereof.

1. A system comprising:
an ion-sensitive sensor that includes a dielectric layer including Al₂O₃;
an electrolytic solution in which the ion-sensitive sensor is immersed, the electrolytic solution containing a concentration of alkali ions, a surface of the dielectric layer of the ion-sensitive sensor being in contact with the electrolytic solution; and
an electrode arranged to apply an electric potential to the surface of the dielectric layer in contact with the electrolytic solution.

2. The system claim 1, wherein the surface of the dielectric layer in contact with the electrolytic solution is a functionalized surface configured to bond with an analyte.

3. The system of claim 2, wherein the ion-sensitive sensor is an ion-sensitive silicon field effect transistor (FET) and the dielectric layer in contact with the electrolytic solution is the gate dielectric layer of the ion-sensitive silicon FET.

4. The system of claim 3, wherein the electrode comprises a perforated gate metal layer disposed on the gate dielectric layer of the ion-sensitive silicon FET, the functionalized surface being disposed in openings of the perforated gate metal layer.

5. The system of claim 3, wherein the electrode comprises a reference electrode immersed in the electrolytic solution but not disposed on the gate dielectric layer of the ion-sensitive silicon FET.

6. The system of claim 2, wherein the functionalized surface of the dielectric layer includes protein receptors.

7. The system of claim 2, wherein the functionalized surface of the dielectric layer including Al₂O₃ is a surface including receptors that selectively bind with an analyte organic molecule.

8. The system of claim 1, wherein the dielectric layer in contact with the electrolytic solution comprises a multi-layer dielectric stack comprising two or more layers including at least one Al₂O₃ layer.

9. The system of claim 8, wherein the multi-layer dielectric stack also includes at least one dielectric layer selected from a group consisting of hafnium silicate, zirconium silicate, hafnium dioxide, zirconium dioxide, tantalum oxide, titanium dioxide, or combinations thereof.

10. The system of claim 8, wherein the multi-layer dielectric stack is deposited by atomic layer deposition (ALD).

11. The system of claim 1, wherein the ion-sensitive sensor is an ion-sensitive silicon field effect transistor (FET), the dielectric layer in contact with the electrolytic solution is the gate dielectric layer of the ion-sensitive silicon FET, and the gate dielectric layer of the ion-sensitive silicon FET is deposited by atomic layer deposition (ALD).

12. The system of claim 1, wherein the ion-sensitive sensor is an ion-sensitive π-conjugated field effect transistor (FET) and the dielectric layer in contact with the electrolytic solution is the gate dielectric layer of the ion-sensitive π-conjugated FET.

13. The system of claim 12, wherein the surface of the dielectric layer in contact with the electrolytic solution is a functionalized surface configured to bond with an analyte, and the electrode comprises a perforated gate metal layer disposed on the gate dielectric layer of the ion-sensitive π-conjugated FET, the functionalized surface being disposed in openings of the perforated gate metal layer.

14. The system of claim 12, wherein the surface of the dielectric layer in contact with the electrolytic solution is a functionalized dielectric surface, including Al₂O₃, configured to bond with an analyte.

15. The system of claim 12, wherein the surface of the dielectric layer in contact with the electrolytic solution is a functionalized surface that includes receptors that selectively bind with an analyte organic molecule.

16. The system of claim 12, wherein the gate dielectric layer comprises a multi-layer dielectric stack comprising two or more layers including at least one Al₂O₃ layer.

17. The system of claim 16, wherein the multi-layer dielectric stack also includes at least one dielectric layer selected from a group consisting of hafnium silicate, zirconium silicate, hafnium dioxide, zirconium dioxide, tantalum oxide, titanium dioxide, or combinations thereof.

18. The system of claim 12, wherein the gate dielectric layer of the ion-sensitive π-conjugated FET is deposited by atomic layer deposition (ALD).

19. The system of claim 12, wherein the ion-sensitive π-conjugated FET is a polymer FET.

20. A method comprising:
depositing a gate dielectric layer comprising Al₂O₃ on a substrate by atomic layer deposition (ALD) to form an ion-sensitive field effect transistor (FET); and
modifying an exposed surface of the deposited gate dielectric layer to generate a functionalized gate dielectric surface configured to bond with an analyte.

21. The method of claim 20 further comprising:
immersing the ion-sensitive FET with the functionalized gate dielectric surface in an electrolytic solution containing a concentration of alkali ions; and
operating the ion-sensitive FET to measure concentration of the analyte in the electrolytic solution, the operating including biasing an electrode arranged to apply an electric potential to the functionalized gate dielectric surface of the ion-sensitive FET.

22. The method of claim 20 wherein the substrate is a silicon substrate and the ion-sensitive FET is an ion-sensitive silicon FET.

23. The method of claim 20 wherein the substrate is a polymer substrate and the ion-sensitive FET is an ion-sensitive π-conjugated FET.

24. A sensor comprising;
an ion-sensitive field effect transistor (FET) or capacitor that includes a dielectric layer comprising Al₂O₃; and
a perforated metal layer disposed on the dielectric layer of the ion-sensitive FET or capacitor;
wherein the dielectric layer includes a functionalized surface configured to bond with an analyte, the functionalized surface being disposed in openings of the perforated metal layer.

25. The sensor of claim 24 wherein the functionalized surface is a functionalized Al₂O₃ surface.

26. The sensor of claim 24 wherein:
the ion-sensitive FET or capacitor is an ion-sensitive FET,
the dielectric layer is the gate dielectric layer of the ion-sensitive FET, and
the metal layer is a gate metal layer disposed on the gate dielectric layer of the ion-sensitive FET.

27. The sensor of claim 26 wherein the ion-sensitive FET is an ion-sensitive silicon FET.

28. The sensor of claim 26 wherein the ion-sensitive FET is an ion-sensitive π-conjugated FET.

29. The sensor of claim 24 wherein the dielectric layer comprising Al₂O₃ comprises:
a multi-layer dielectric stack comprising two or more layers including at least one Al₂O₃ layer.
